(19) United States
(12) Patent Application Publication
(10) Pub. No.: US 2004/0185128 A1
(43) Pub. Date: Sep. 23, 2004

(54) Use of an extract of the root of balloon-flower for preventing and treating a degenerative brain disease or for enhancing memory

(76) Inventors: Young-Sup Kim, Daejeon (KR); Shi-Young Ryu, Daejeon (KR); Sung-Ki Kim, Daejeon (KR); Jung-Hee Heor, Daejeon (KR); Jong-Seong Kang, Daejeon (KR)

Correspondence Address: David A Einhorn, Anderson Kill & Olick, 1251 Avenue of the Americas, New York, NY, 10020, US

(21) Appl. No.: 10/480,523

(22) PCT Filed: Jun. 15, 2002
(86) PCT No.: PCT/KR02/01129

(30) Foreign Application Priority Data
Jun. 15, 2001 (KR) 2001-0033779

(51) Int. Cl. A61K 35/78

(52) U.S. Cl. 424/773; 424/764; 424/739; 424/757

(57) ABSTRACT

Disclosed in this invention is a use of an extract of the root of balloon-flower for preventing and treating a degenerative brain disease or for enhancing memory.

### FIELD OF THE INVENTION

The present invention relates to a use of an extract of the root of balloon-flower for preventing or treating degenerative brain diseases or enhancing memory.

### BACKGROUND OF THE INVENTION

An ever increasing number of the elderly population afflicted by degenerative brain diseases such as senile dementia, Parkinson's disease, cerebral apoplexy, and Huntington's disease has become a major social problem particularly because no effective drugs or methods for preventing and treating such diseases are presently available.

Senile dementia, a representative degenerative brain disease, is usually preceded by chronic or progressive degeneration of brain cells and shows impairment in the cognitive capacity which controls memory, thinking, comprehension, calculation, learning, language and judgment.

The exact cause of senile dementia has not been yet elucidated, however, it has been reported to be caused by damage of cholinergic neurons in the cerebral base, reduction of neurotransmitter, deposition of β-amyloid protein due to inflammatory reaction, and oxidative stress (Davies P., et al., Lancet, 21, 1403 (1976); Rocher, A. E., et al., J. Biol. Chem., 273, 29719 (1988); and Coyle, J. T., et al. Science, 262, 689 (1993)).

As present, protecting or restoring neurons is considered to provide a viable method for treating senile dementia, but a pharmaceutical composition therefor has not yet been developed.

The present inventors have endeavored to develop an effective drug of a natural origin for preventing and treating degenerative brain diseases, and, as a result, have discovered that an extract of the root of Balloon-flower suppresses cholinergic nerve cell damage by inhibiting overexpression of irritable neurotransmitter glutamate, and increases the cognitive and learning capacities by enhancing the cholinergic neurotransmitter activity.

Balloon-flower (Platycodon gradiflorum A. DC) has long been used as an edible vegetable and for medicinal purposes. It has been reported that terpenoid saponin, a major active component of balloon-flower, is effective as an antitussive agent, expectorant, central nerve inhibitor (sedation, analgesia and antipyretic action), anti-inflammatory agent on acute and chronic inflammation, anti-ulcer agent and anti-sialic agent of gastric juice, anti-choline agent which reduces the cholesterol level by enlarging blood vessels, hypoglycemic agent and cholesterol metabolism modifier activities (Toshiyuki Akiyama et al., Chem. Pharm. Bull., 20, 1952 (1972); Akihito Tada. et al., Chem. Pharm. Bull., 23, 2965 (1975); Hiroshi Ishii et al., J. Chem. Soc., Perkin trans I, 661(1984); and Eun Bang Lee, J. Pharm. Soc. Kor., 19, 164(1975)).

Further, it has been reported that a hot water or ethanol extract of the balloon-flower suppresses the aflatoxin of fungi; the inulin fraction thereof has a phagocytic effect and anti-tumor activity on solid and ascites cancers; and a 40% balloon-flower extract concentrate suppresses on alcohol absorption (Hitokoto H. S. et al., Mycopathologia, 66, 16(1979); Michinori Kubo, et al., Shoyakugaku Zasshi, 40 367(1986); Takaharu Nagao et al., Shoyakugaku Zasshi, 40, 375(1986); and JPA 3-264534 (1991)).

In spite of the above mentioned efficacies of balloon-flower extract, the development thereof as therapeutic medicines has been hampered due to difficulties in cultivating the plant. However, Sung Ho Lee has recently reported on a method of growing more than 20-year-old Balloon-flower (Sung Ho Lee, “Growing method of the perennial Balloon-flower”, Korean Patent No. 045791), which triggered numerous efforts to develop medicines therefrom.

Further, it has been reported that the root extract of 20-year-old or more than 20-year-old balloon-flower, called “long-life (Jang Saeng)” balloon-flower, is effective in hyperlipidemia treatment (Kyung-sook Kim et al., J. Nur. Sci. Vitamino, 41, 485 (1995)), protecting liver (Jeong H. K., et al., Cancer Letters, 174, 73 (2001)), and controlling the immune system (Sang B. Han et al., International Immunopharmacology, 1, 1969(2001); Jeong H. K. et al., Cancer Letters, 166, 17(2001); and Jeong H. K. et al., International Immunopharmacology, 1, 1141(2001)).

The present inventors have unexpectedly found that a root of balloon-flower extract has pronounced effects in preventing or treating degenerative brain diseases and enhancing memory.

### SUMMARY OF THE INVENTION

Accordingly, it is an object of the present invention to provide a pharmacologically active substance for preventing and treating degenerative brain diseases.

It is another object of the present invention to provide a pharmacologically active substance for enhancing memory.

### DETAILED DESCRIPTION OF THE INVENTION

In accordance with one aspect of the present invention, there is provided a use of an extract of the root of balloon-flower for preventing or treating a degenerative brain disease in a mammal.

In accordance with another aspect of the present invention, there is provided a use of an extract of the root of balloon-flower for enhancing memory in a mammal.

The root of balloon-flower which may be used in the present invention is inclusive of Platycodon gradiflorum A. DC, Platycodon grandiflorum for albiflorum Hara and the like, and preferably more than 20-year-old long-life balloon-flower.

The extract of the root of balloon-flower of the present invention can be prepared by extracting with water or an organic solvent, e.g., a lower alcohol, acetone, chloroforum, methylenechloride, ether, ethylacetate, and hexane. Examples of the lower alcohol are methanol, ethanol, propanol and butanol, preferably ethanol.

The balloon-flower root used in the extraction procedure of the present invention may be in a raw, dried, or powder form, preferably a powder form, and more preferably a dried balloon-flower root powder having a moisture content of less than 5% and an average size of less than 0.6 mm.

Specifically, a hot-water extract of the root of balloon-flower can be prepared by adding 5 to 15 fold volume of water, preferably a 10-fold volume of water to a dried balloon-flower powder and extracting for 1 to 24 hours, preferably 4 to 6 hours at 80 to 100° C., preferably 90 to 95° C., and then filtered. Alternately, 1 to 15-fold volume, preferably 3-fold volume of an organic solvent may be used to extract a balloon-flower root powder at room temperature, to obtain an organic solvent extract. The above extraction procedure may be repeated two more times as needed. Also, after the filtration, a powder form of the extract can be prepared by removing the solvent of the extract under a reduced pressure.

In order to prevent and treat degenerative brain diseases, or to enhance memory, the extract of balloon-flower root can be administered to a mammal in the form of a composition containing, e.g., a pharmaceutical composition, a food composition or a beverage composition.

The pharmaceutical composition of the present invention may additionally include a pharmaceutically acceptable medicinal herb medicines or an extract thereof for the purpose of enhancing the intended effect. In this case, a herb extract prepared according to the above extraction procedure, or an extract of a mixture of balloon-flower root and one or more herbs prepared according to the above extraction procedure may be used.

The herb which may be suitably used in the composition of the present invention is any of pharmaceutically acceptable herbs. Examples of such herbs are Angelicae tenuissimae Radix, Gastrodiae Rhizoma, Bupleuri Radix, Angelicae gigantis Radix, Persicae Semen, Cinnamomi Ramulus, Rhei Rhizoma, Glycyrrhizae Radix, Cnidii Rhizoma, Aurantii nobilis Pericarpium, Alismatis Rhizoma, Coptidis Rhizoma, Scutellariae Radix, Hoelen, Paeoniae Radix, Atractylodis Rhizoma alba, Phellodendri Cortex, Gardeniae Fructus, Pinelliae Tuber, Uncaria Ramulus et Uncus, Ponciri Fructus, Ginseng, Liriopis Tuber, Polygalae Radix, Acori graminei Rhizoma, Atractylodis Rhizoma alba, Chrysanthemi Flos, Ledebouriellae Radix, Zingiberis Rhizoma crudus, Zizyphi Fructus, Salviae Radix, Persicae Semen, Moutan Radicis Cortex, Rehmanniae Radix, Menthae Herba, Dioscoreae Rhizoma, Polyporus, Polygoni multiflori Radix, Allii tuberosi Semen, Cassiae Semen, Lycii Fructus, Araliae cordatae Radix, Eucommiae Cortex, Hedyotis Herba, Saururus Herba, Artemisiae capillaries Herba, Anemarrhenae Rhizoma, Carthami Flos, Astragali Radix, Lycopodium, Ginkgonis Folium, Polygonati Rhizoma, Nelumbinis Semen, Fossilia ossis. Mastodi, Lycii radicis Cortex, Achyranthis Radix, Rehmanniae Radix preparata, Perillae Semen, Thujae Semen, Hordei Fructus germinatus, Cuscutae Semen, Morindae Radix, Pini koraiensis Radix and a mixture thereof, preferably, Gastrodiae Rhizoma, Angelicae gigantis Radix, Cnidii Rhizoma, Alismatis Rhizoma, Coptidis Rhizoma, Hoelen, Paeoniae Radix, Atractylodis Rhizoma alba, Polygalae Radix, Moutan Radicis Cortex, Dioscoreae Rhizoma, Polyporus, Allii tuberosi Semen, Lycopodium, and Ginkgonis Folium.

The content of the balloon-flower root extract in the pharmaceutical composition of the present invention may range form 10 to 100 wt %, preferably 30 to 70 wt % based on the total weight of the composition, and the amount of the herb or an extract thereof in the pharmaceutical composition of the present invention may range form 0 to 90 wt %, preferably 30 to 70 wt % based on the total weight of the composition.

The pharmaceutical composition of the present invention can effectively suppress cranial nerve cell damage caused by overflow of irritable neurotransmitter glutamate, by way of inhibiting glycine binding site of glutamate receptor, and also can prevent the loss of cognitive ability by promoting the cholinergic neurotransmitter activity in muscarinic receptor; therefore, the pharmaceutical composition of the present invention exerts superior preventive and treating effects on degenerative brain diseases such as senile dementia, Parkinson's disease, cerebral apoplexy, Huntington's disease and the like.

Further, the pharmaceutical composition of the present invention enhances learning ability and memory as shown in an animal tesst

Moreover, in spite of its potent efficacies, the pharmaceutical composition containing the balloon-flower extract shows little toxicity or mitogenicity in test using mice and exert no adverse effects on the liver function.

A pharmaceutical formulation may be prepared in accordance with any of the conventional procedures. In preparing the formulation, the active ingredient is preferably admixed or diluted with a carrier, or enclosed within a carrier, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, calcium silicate, cellulose, methyl cellulose, microcrystalline cellulose, polyvinylpyrrolidone, water, methylhydroxybenzoates, propylhydroxybenzoates, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction. In case of human, a typical daily dose of the Balloon-flower extract may range from about 1 to 1,000 mg/kg body weight, preferably 10 to 100 mg/kg body weight, and can be administered in a single dose or in divided doses. However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The present invention also provides a method for preventing or treating degenerative brain diseases in mammals, which comprises administering thereto an effective amount of the balloon-flower extract and an additional herbal extract. Further, the present invention provides a method for enhancing memory in mammals, which comprises administering thereto an effective amount of the balloon-flower extract and an optional herbal extract.

Moreover, the balloon-flower extract and the additional herbal extracts can be incorporated in foods or beverages, as an additive or a dietary supplement, for the purpose of preventing degenerative brain diseases of various kinds or improving memory. In this case, the content of the Balloon-flower extract in a food or beverage may range from 0.1 to 15 wt %, preferably 1 to 10 wt % based on the total weight of the food, and 1 to 30 g, preferably 3 to 10 g of per 100 ml of the beverage.

The health care beverage composition of the present invention may contain other components, e.g., deodorants and natural carbohydrates as in conventional beverages. As the deodorant, a natural deodorant such as taumatin, Stevia extract, e.g., levaudioside A, glycyrrhizin and the like, or a synthetic deodorant such as saccharin and aspartam may be used. Examples of such natural carbohydrates are monosaccharides such as glucose and fructose; disaccharides such as maltose and sucrose; conventional polysaccharides such as dextrin and cyclodextrin; and sugar alcohols such as xylitol, sorbitol and erythritol. The amount of the above-described natural carbohydrate is generally in the range of about 1 to 20 g, preferably 5 to 12 g based on 100 ml of beverage.

Other components that may be added to the inventive food or beverage composition are various nutrients, vitamins, minerals, synthetic flavoring agents, coloring agents, pectic acid and its salt, alginic acid and its salt, organic acids, protective colloidal adhesives, pH controlling agents, stabilizers, preservatives, glycerin, alcohol, carbonizing agents used in carbonated beverage. The amount of the above-described additives is generally in the range of about 0 to 20 weight portions based on 100 weight portions of the composition.

Moreover, the foods containing the Balloon-flower extract and the additional herbal extracts to develop health supplementary food, may include various foods, various beverages, various gums, vitamin complexes.

The following examples are intended to further illustrate the present invention without limiting its scope.

Also, in the examples below, the percentage with respect to the solid/solid mixture, liquid/liquid, and solid/liquid is each considered at weight/weight, volume/volume, and weight/volume, respectively and unless it is specifically instructed, all experiments are carried out at room temperature.

### EXAMPLE 1

### Preparation of Herbal Extracts and Pharmaceutical Compositions Containing Same

(1) Preparation of Hot Water Extracts

0.2 kg of a “long life (Jang Saeng)” Balloon-flower root (Jang Saeng Doraji Inc.) powder was extracted twice with 2 l portions of the distilled water at 90 to 95° C. for 5, hours, the extract solutions were combined, filtered, and water was removed to obtain 80 g of a Jang Saeng Balloon-flower extract.

Also, the herbal mixtures listed in Table 1a and 1b were each extracted according to the same procedure and pharmaceutical compositions were prepared by mixing the Jang Saeng extracts thus obtained.

(2) Preparation of Ethanol Extracts

Jang Saeng Balloon-flower and herbs were mixed as listed in Table 1a and 1b, and each mixture was extracted twice with 3-fold volume of ethanol at room temperature, the extract solutions were combined, filtered, and the filtrate was concentrated under a reduced pressure, to obtain a pharmaceutical composition.

TABLE 1a

| | |
|---|---|
| AL-1 | Platycodi Radix (20) |
| AL-2 | Platycodi Radix (20), Angelicae tenuissimae Radix (3), Gastrodiae Rhizoma (3), |
| | Alismatis Rhizoma (6), Cnidii Rhizoma (6), Angelicae gigantis Radix |
| | (3), Hoelen (3), Atractylodis Rhizoma alba (3), Paeoniae Radix (10) |
| AL-3 | Platycodi Radix (20), Angelicae tenuissimae Radix (3), Gastrodiae Rhizoma |
| | (3), Coptidis Rhizoma (5), Scutellariae Radix (5), Phellodendri Cortex (5), |
| | Gardeniae Fructus (5) |
| AL-4 | Platycodi Radix (20), Angelicae tenuissimae Radix (3), Gastrodiae Rhizoma |
| | (3), Bupleuri Radix (2), Angelicae gigantis Radix (3), Atractylodis Rhizoma |
| | alba (4), Hoelen (4), Glycyrrhizae Radix (1.5), Cnidii Rhizoma (3), Uncaria |
| | Ramulus et Uncus (3), Aurantii nobilis Pericarpium (3), Pinelliae Tuber (5) |
| AL-5 | Platycodi Radix (20), Angelicae tenuissimae Radix (3), Gastrodiae Rhizoma |
| | (3), Chrysanthemi Flos (3), Liriopis Tuber (3), Pinelliae Tuber (3), Aurantii |
| | nobilis Pericarpium (3), Hoelen (3), Panax ginseng (2), Uncaria Ramuluset |
| | Uncus (3), Zingiberis Rhizoma crudus (1), Glycyrrhizae Radix (1), |
| | Ledebouriellae Radix (2) |
| Al-6 | Platycodi Radix (20), Angelicae tenuissimae Radix (3), Gastrodiae Rhizoma |
| | (3), Persicae Semen (5), Cinnamomi Ramulus (4), Rhei Rhizoma (1.5), |
| | Glycyrrhizae Radix (1.5), Natrii sulfas (0.9) |
| AL-7 | Platycodi Radix (20), Angelicae tenuissimae Radix (3), Gastrodiae Rhizoma |
| | (3), Bupleuri Radix (16), Pinelliae Tuber (4), Scutellariae Radix (10), |
| | Paeoniae Radix (10), Zizyphi Fructus (2), Ponciri Fructus (6), Zingiberis |
| | Rhizoma crudus (1), Rhei Rhizoma (4) |
| AL-8 | Platycodi Radix (20), Angelicae tenuissimae Radix (3), Gastrodiae Rhizoma |
| | (3), Coptidis Rhizoma (3), Scutellariae Radix (3), Rhei Rhizoma (3) |
| AL-9 | Platycodi Radix (20), Angelicae tenuissimae Radix (3), Gastrodiae Rhizoma |
| | (3), Cinnamomi Ramulus (3), Paeoniae Radix (3), Hoelen (3), Persicae |
| | Semen (3), Moutan Radicis Cortex (3), Salviae Radix (5), Carthami Fols (3) |
| AL-10 | Platycodi Radix (20), Angelicae tenuissimae Radix (3), Gastrodiae Rhizoma |
| | (3), Paeoniae Radix (3), Rehmanniae Radix (3), Angelicae gigantis Radix |
| | (3), Cnidii Rhizoma (3) |

TABLE 1b

| | |
|---|---|
| AL-11 | Platycodi Radix (20), Angelicae tenuissimae Radix (3), Gastrodiae Rhizoma |
| | (3), Angelicae gigantis Radix (3), Paeoniae Radix (3), Bupleuri Radix (3), |
| | Atractylodis Rhizoma alba (3), Hoelen (3), Menthae Herba (1), Moutan |
| | Radicis Cortex (2), Gardeniae Fructus (2), Glycyrrhizae Radix (2), Zingiberis |
| | Rhizoma crudus (1) |
| AL-12 | Platycodi Radix (20), Hoelen (4), Polygalae Radix (4), Acori graminei |
| | Rhizoma (4) |
| AL-13 | Platycodi Radix (20), Hoelen (20), Acori graminei Rhizoma (3), Polygoni |
| | multiflori Radix (6), Polygalae Radix (3), Dioscoreac Rhizoma (9), Coptidis |
| | Rhizoma (6), Gastrodiae Rhizoma (3), Angelicae tenuissimae Radix (3), |
| | Polyporus (6), Cinnamomi Ramulus (1.5) |
| AL-14 | Platycodi Radix (20), Angelicae tenuissimae Radix (3), Gastrodiae Rhizoma |
| | (3), Alismatis Rhizoma (6), Cnidii Rhizoma (6), Angelicae gigantis Radix |
| | (3), Hoelen (3), Atractylodis Rhizoma alba (3), Paeoniae Radix (10), Coptidis |
| | Rhizoma (5), Dioscoreae Rhizoma (4), Allii tuberosi Semen (5), Polyporus |
| | (10), Phellodendri Cortex (5) |
| Al-15 | Platycodi Radix (20), Glycyrrhizae Radix (6.7), Cassiae Semen (5.6), Lycii |
| | Fructus (5.6), Astragali Radix (5.6), Anemarrhenae Rhizoma (7.8), Araliae |
| | cordatae Radix (5.6), Ledebouriellae Radix (5.6), Cinnamomi Ramulus (5.6), |
| | Atractylodis Rhizoma alba (5.6), Artemisiae capillaris Herba (1.1), |
| | Eucommiae Cortex (5.6), Dioscoreae Rhizoma (22), Carthami Fols (7.2), |
| | Saururus Herba (5), Hedyotis Herba (1.7) |
| AL-16 | Platycodi Radix (20), Polygalae Radix (6), Gastrodiae Rhizoma (4), Alismatis |
| | Rhizoma (6), Cnidii Rhizoma (6), Angelicae gigantis Radix (6), Hoelen (3), |
| | Atractylodis Rhizoma alba (3), Paeoniae Radix (5), Coptidis Rhizoma (5), |
| | Dioscoreae Rhizoma (5), Allii tuberosi Semen (5), Polyporus (10), |
| | Lycopodium (10), Moutan Radicis Cortex (8), Ginkgonis Folium (10) |
| AL-17 | Platycodi Radix (20), Polygalae Radix (9), Coptidis Rhizoma (5), Dioscoreae |
| | Rhizoma (10), Allii tuberosi Semen (5), Angelicae gigantis Radix (10), |
| | Hoelen (10), Polyporus (10), Lycopodium (10), Moutan Radicis Cortex (8), |
| | Ginkgonis Folium (10) |
| AL-18 | Platycodi Radix (20), Polygonati rhizoma (4), Dioscoreae hizoma (8), |
| | Nelumbinis semen (4), Fossilia ossis mastodi (4), Polygalae radix (8), Lycii |
| | radicis cortex (4), Lycii fructus (4), Acori graminei rhizoma (8), Eucommiae |
| | cortex (4), Achyranthis radix (4), Rehmanniae radix preparata (4), Perillae |
| | semen (4), Gastrodiae rhizoma (8), Thujae semen (3), Hordei fructus |
| | germinatus (3), Cuscutae semen (6), Angelicae gigantis radix (6), Morindae |
| | radix (6), Ginseng radix (6), Pini koraiensis radix (4), Angelicae tenuissimae |
| | Radix (4) |
| AL-19 | Platycodi Radix (20), Polygonati rhizoma (4), Dioscoreae hizoma (8), |
| | Nelumbinis semen (4), Fossilia ossis mastodi (4), Polygalae radix (10), Lycii |
| | radicis cortex (4), Lycii fructus (4), Acori graminei rhizoma (8), Eucommiae |
| | cortex (4), Achyranthis radix (4), Rehmanniae radix preparata (4), Perillae |
| | semen (4), Gastrodiae rhizoma (12), Thujae semen (3), Hordei fructus |
| | germinatus (3), Cuscutae semen (6), Angelicae gigantis radix (10), Morindae |
| | radix (6), Ginseng radix (6), Pini koraiensis radix (8), Angelicae tenuissimae |
| | Radix (4) |

### EXAMPLE 2

### Effect of Increasing the Activity of Cholinergic Neurotransmitter, Acetylcholine

The ability of each of the compositions obtained in Example 1 in enhancing acetylcholine activity was measured by examining to what extent the binding of a ligand to muscarin acetylcholine receptor subtype 1(M₁) is suppressed. That is, an excess amount of a radioactive isotope-labeled ligand was allowed to react with the receptor, unbound ligand was removed by filtering using a glass fiber filter and the amount of the isotope-labeled ligand on the filter was measured to quantify the amount of ligand bound to receptor, and thus, the effects of the compositions of the present invention was determined.

As the receptor, recombinant human muscarinic acetylcholine receptor subtype 1(mAChR-M₁, BSR-MM1H, BSR) expressed in Chinese Hamster Ovary (CHO) cells was used. 250 μl of a deep-frozen (−70° C.) receptor fraction was suspended in 10 ml of phosphate buffer saline (PBS) (pH 7.4) and the concentration of the protein was adjusted to 130 μg/ml.

50 μl of 0.5 nM [³H] N-methyl-scopolamine (24,605 DPM) (NEN, NET-636) and 10 μl of a test composition were added to each well of 96-well microtiter plate (Inotech harvester). In order to correct for non-specific binding, 50 μl of 5 μM atropine sulfate was added thereto. Then, 100 μl of the receptor suspension obtained above was added to each well and final volume was adjusted to 0.25 ml with PBS. The resulting mixture was reacted at 25° C. for 60 minutes while shaking. The reaction was terminated by adding 0.5 μml of 50 mM Tris-HCl buffer solution/0.9% cold saline (pH 7.4), immediately filtered with Inotech cell harvester system using Wallac glass fiber filtermat GF/C (Wallac, P.O. Box 10, FIN-20101 Tutku, Finland), and then washed 3 times with cold PBS. The filtermat was dried in a microwave oven and the amount of the ligand bound to the receptor was evaluated by determining the radioactivity with the liquid scintillation counter (MicroBeta 1450 Plus; Wallac, Finland). Each composition obtained in Example 1 was diluted with PBS containing a small amount of dimethylsulfoxide (DMSO), and the DMSO concentration of the reaction solution was adjusted to less than 0.1%. The assay was repeated twice to determine an average value. The inhibiting activity % calculated based on the result for a control was determined and the result is shown in Table 2. As a control, 4-DAMP methiodide which inhibited the ligand binding to receptor by 50% at 0.024 μM was used

TABLE 2

| | Degree of |
|---|---|
| | receptor binding |
| | inhibition |
| Composition No. | 0.5(mg/ml), % |
| AL-1 | <0.0 |
| AL-2 | <0.0 |
| AL-3 | 87.39 |
| AL-4 | <0.0 |
| AL-5 | <0.0 |
| AL-6 | <0.0 |
| AL-7 | <0.0 |
| AL-8 | 18.49 |
| AL-9 | <0.0 |
| AL-10 | <0.0 |
| AL-11 | <0.0 |
| AL-12 | <0.0 |
| AL-13 | 69.4 |
| AL-14 | 71.2 |
| AL-15 | 40.9 |
| AL-16 | 52.2 |
| AL-17 | 25.3 |
| AL-18 | 39.8 |
| AL-19 | 40.8 |

As can be seen in Table 2, the muscarine receptor-ligand binding was inhibited by AL-3, AL-13, AL-14, and AL-16 at a respective concentration of 0.5 mg/ml, while AL-15, Al-18, and AL-19 also showed relatively high inhibiting activity.

Accordingly, it has been confirmed that the pharmaceutical composition of the present invention can effectively inhibit the binding of the ligand to muscarin receptor, and thus, improves the efficacy of the brain cholinergic neurotransmitter and enhances the cognitive function.

### EXAMPLE 3

### Inhibition of Neuron Damage

The fact that the compositions of the present invention suppresses neuron damage was confirmed as follows by examining the activity thereof in suppressing the binding formation of NMDA-receptor (glycine site) bound. NMDA (N-Methyl-D-Aspartate) which acts as an excitatory neurotransmitter induces neuron damage.

That is, an excess amount of a radioactive isotope-labeled ligand was allowed to react with the receptor, unbound ligand was removed by filtering using a glass fiber filter and the amount of the isotope-labeled ligand on the filter was measured to quantify the amount of ligand bound to receptor, and thus, the effects of the compositions of the present invention was determined.

(Step 1) Preparation of NMDA Receptor Fraction from Rat's Cerebrum

Forebrain taken from a male Spargue-Dawley rat was sliced and a 10-fold volume of cold sucrose solution (0.32 mM) was added thereto. The resulting mixture was homogenized (5 strokes) using a Teflon-glass homogenizer, and then centrifuged at 1000 g (10 min., 4° C.). The supernatant was centrifuged at 20000 g (20 min., 4° C.) to obtain a precipitate, a 20-fold volume of cold distilled water was added thereto and homogenized using Brinkman Polytron Homogenizer. The homogenate thus obtained was stirred at 4° C. for 30 minutes and centrifuged at 8000 g (20 min., 4° C.). The supernatant thus obtained was centrifuged at 39,800 g (25 min., 4° C.) and then the precipitate thus obtained was stored in a deep-freezer of −70° C. The deep-freezed precipitate was thawed at room temperature for 10 minutes and suspended in 50 mM tris-acetate buffer solution (pH 7.1) containing a 20-fold volume of 0.04% triton X-100. The resulting mixture was stirred at 37° C. for 20 minutes and centrifuged at 39800 g (20 min., 4° C.). The precipitate thus obtained was washed 3 times, eachtime by resuspended in a 20-fold volume of 50 mM tris-acetate buffer solution (pH 7.1), and centrifuged, and then suspended in the same buffer solution. The protein content was measured according to Bradford method and the protein concentration of suspension was adjusted to 1 mg/ml, divided into several fractions and kept at −70° C.

(Step 2) Test Suppressing Neuron Damage

The receptor cell fraction kept at −70° C. was suspended in 50 mM tris-acetate buffer solution (pH 7.1).

50 μl of 4 nM [³H]MDL 105,519 (140,000 DPM, Amersham Pharmacia Biotech) and 10 μl of the test composition were added to each well of a 96-well microtiter plate (Inotech harvester). In order to correct for non-specific binding, 50 μl of 5 mM glycine was added thereto. 100 μl of the receptor suspension obtained above was added to each well and the final volume was adjusted to 0.25 ml with 50 mM tris-acetate. The receptor protein content in the reaction solution was 5 μg/well. The resulting solution was reacted at 25° C. for 30 minutes while shaking, and the reaction was terminated by adding 0.2 ml of 50 mM Tris-HCl buffer solution/0.9% cold saline (pH 7.4). The resulting solution was immediately filtered with Inotech cell harvester system using Wallac glass fiber filtermat GF/C (Wallac, P.O. Box 10, FIN-20101 Tutku, Finland). The filtrate was washed 9 times with cold 50 mM Tris-acetate buffer, dried in a microwave oven and the extract of the ligand binding to the receptor was evaluated by determining the radioactivity with a liquid scintillation counter (MicroBeta 1450 Plus; Wallac, Finland). Each composition obtained in Example 1 was diluted with PBS containing a small amount of dimethylsulfoxide (DMSO), and the DMSO concentration of the reaction solution was adjusted to less than 0.1%. The assay was repeated twice to determine an average value. The inhibiting activity % calculated based on the result for a control was determined and the result is shown in Table 3. As a control, 5,7-DCKA (5,7-Dichlorokynurenic acid. RBI) which inhibited the ligand binding to receptor by 50% at 1.0 μM was used

TABLE 3

| Composition No. | IC50(mg) |
|---|---|
| AL-1 | 0.051 |
| AL-2 | 0.015 |
| AL-3 | 0.052 |
| AL-4 | 0.024 |
| AL-5 | 0.051 |
| AL-6 | 0.031 |
| AL-7 | 0.041 |
| AL-8 | 0.033 |
| AL-9 | 0.025 |
| AL-10 | 0.039 |
| AL-11 | 0.044 |
| AL-12 | 0.056 |
| AL-13 | 0.057 |
| AL-14 | 0.056 |
| AL-15 | 0.048 |
| AL-16 | 0.048 |
| AL-17 | 0.050 |
| AL-18 | 0.038 |
| AL-19 | 0.041 |

As can be seen from Table 3, the inventive pharmaceutical composition at a concentration of 15 to 50 μg, strongly suppresses the ligand binding on the glycine binding site of the NMDA receptor.

Accordingly, it has been confirmed that the pharmaceutical composition of the present invention can effectively deactivate the glycine-binding site of the NMDA receptor, and thus, can prevent the loss of the cognitive capacity by suppressing the production of excitatory neurotransmitter,

### EXAMPLE 4

### Passive-avoidance Test

(1) Test Method

Male rats, each weighing about 18 to 20 g, were raised under a condition of temperature 22±1° C. and 12L/12D photoperiod for 7 days while being allowed free access to food and water and used in the Test after 3 days of acclimatization. The rats were divided into 3 groups and administered daily with 3 different compositions over a period of week; 250 mg/kg Tween 80 (polyoxyethylenesorbitan monooleate, Sigma) containing the pharmaceutical composition prepared in Example 1 (Test group); 2.5 mg/kg of Tacrine (9-amino-1,2,3,4-tetrahydroacrine: Sigma) (Comparative group); and 5% Tween (Control group), respectively.

The passive avoidance test composed of consecutively learning and testing procedures was conducted over a period of 2 days (interval: 24hours) using PACS-30 shuttle box system (Columbus Instrument Co.).

(1-1) Learning Procedure

30 minutes after the 7^{th} (last) administeration, a 3 g/kg dose of 50% ethanol was orally administered to respective rats. After 1 hour, the rats were placed in the bright area of a room which was divided into a dark and bright areas by a guillotin door and allowed to stay there for 30 seconds (the searching time). The guillotin door was opened to let the rats move to the dark side of the room. The rats which did not move to the dark side of the room within 120 seconds after opening of the guillotin door were rejected from the experiment. The time the rats took to move from the bright side to the dark side was measured automatically. The guillotin door was closed shut as soon as the test subject move to the dark side, and then 0.4 mA of scramble shock was applied through the grid floor for 5 seconds for the rat to remember same.

(1-2) Testing Procedure

24 hours after the learning procedure, test procedure was conducted as follows. After 30 seconds of the searching time and opening of the gillontin door, the time the test animal took to move from the bright side to the dark side (latency time) was measured up to the extent 300 seconds. The result is shown in Table 4. Longer the latency time, better the learning ability and memory of the test animal.

(2) Test Result

As can be seen from Table 4, the compositions of the present invention remarkably improve the rats' memory as compared to those of the control and comparative groups.

TABLE 4

| | Latency | | |
|---|---|---|---|
| Test group | Time (s) | Test Substance | Latency Time (s) |
| Control group | 20 | AL-13 | 26 |
| Comparative group | 31 | AL-14 | 131 |
| AL-1 | 96 | AL-15 | 31 |
| AL-2 | 101 | AL-16 | 233 |
| AL-4 | 236 | AL-17 | 194 |
| AL-6 | 236 | AL-18 | 13 |
| AL-12 | 257 | AL-19 | 34 |

### Formulation Examples

The composition of the present invention can be used in preparing a pharmaceutical formulation by only or admixing with pharmaceutical excipients in various pharmaceutical forms according to any one of the conventional methods, as exemplified below without limiting the scope of the present invention.

<Formulation Example 1> Preparation of Powder

| | |
|---|---|
| Dried extract of AL-1 | 2 g |
| Lactose | 1 g |

The above ingredients were mixed thoroughly and then, filled and sealed in a sealed package to obtain a powder preparation.

<Formulation Example 2> Preparation of Tablet

| | |
|---|---|
| Dried extract of AL-16 | 100 mg |
| Corn Starch | 100 mg |
| Lactose | 100 mg |
| Steric Acid Magnesium | 2 mg |

The above ingredients were mixed thoroughly and tabletted according to a conventional method to obtain a tablet preparation.

<Formulation Example 3> Preparation of Capsule

| | |
|---|---|
| Dried extract of AL-1 | 100 mg |
| Corn Starch | 100 mg |
| Lactose | 100 mg |
| Steric Acid Magnesium | 2 mg |

The above ingredients were mixed thoroughly and filled in a gelatin capsule according to a conventional method to obtain a capsule preparation.

<Formulation Example 4> Preparation of Injection Solution

| | |
|---|---|
| Dried extract of AL-16 | 100 mg |
| Distilled water for injection | q.s. |
| pH adjuster | q.s. |

The above ingredients were dissolved in distilled water for injection, and adjusted to pH approximately 7.5. The resulting solution was filled in 2 ml of ample with distilled water for injection and sterilized according to a conventional method to obtain an injection preparation.

<Preparation of Health Care Beverage>

1-10 wt % of AL-1 prepared in Example 1, 5-10 wt % of sugar, 0.05-0.3 wt % of citric acid, 0.005-0.02 wt % of caramel and 0.1-1 wt % of vitamin C were mixed and distilled water was added thereto to obtain a syrup. The syrup thus obtained was sterilized at 85-98° C. for 20-180 seconds and mixed with cooling water to the ratio of 1:4 (v/v). Added thereto was 0.5 to 0.82% of carbonic acid gas to obtain a carbonated beverage containing the extract of Balloon-flower.

Also, AL-16 prepared in Example 1 was homogeneously mixed with liquid fructose (0.5%), oligosaccharide (2%), sugar (2%), saline (0.5%) and water (75%) and instantaneously sterilized to obtain a health beverage.

While the invention has been described with respect to the above specific embodiments, it should be recognized that various modifications and changes may be made to the invention by those skilled in the art which also fall within the scope of the invention as defined by the appended claims.

What is claimed is:

1. A use of a root extract of balloon-flower for preventing or treating a degenerative brain disease in a mammal.

2. The use of claim 1, wherein the mammal is human.

3. The use of claim 1, wherein the balloon-flower is more than 20-year-old long-life balloon-flower.

4. The use of claim 1, wherein the extract is a water extract or an organic solvent extract.

5. The use of claim 4, wherein the water extract is prepared by adding 5 to 15-fold volume of water to a balloon-flower powder; extracting at 80 to 100° C. for 1 to 24 hours; and filtering the extract thus obtained.

6. The use of claim 1, wherein the extract is administered to the mammal in the form of a composition containing same, said composition being selected from the group consisting of: a pharmaceutical composition, a food composition and a beverage composition.

7. The use of claim 6, wherein the composition further comprises a herb or an extract thereof.

8. The use of claim 7, the herb is selected from the group consisting of Angelicae tenuissimae Radix, Gastrodiae Rhizoma, Bupleuri Radix, Angelicae gigantis Radix, Persicae Semen, Cinnamomi Ramulus, Rhei Rhizoma, Glycyrrhizae Radix, Cnidii Rhizoma, Aurantii nobilis Pericarpium, Alismatis Rhizoma, Coptidis Rhizoma, Scutellariae Radix, Hoelen, Paeoniae Radix, Atractylodis Rhizoma alba, Phellodendri Cortex, Gardeniae Fructus, Pinelliae Tuber, Uncaria Ramulus et Uncus, Ponciri Fructus, Ginseng, Liriopis Tuber, Polygalae Radix, Acori graminei Rhizoma, Atractylodis Rhizoma alba, Chrysanthemi Flos, Ledebouriellae Radix, Zingiberis Rhizoma crudus, Zizyphi Fructus, Salviae Radix, Persicae Semen, Moutan Radicis Cortex, Rehmanniae Radix, Menthae Herba, Dioscoreae Rhizoma, Polyporus, Polygoni multiflori Radix, Allii tuberosi Semen, Cassiae Semen, Lycii Fructus, Araliae cordatae Radix, Eucommiae Cortex, Hedyotis Herba, Saururus Herba, Artemisiae capillaries Herba, Anemarrhenae Rhizoma, Carthami Flos, Astragali Radix, Lycopodium, Ginkgonis Folium, Polygonati Rhizoma, Nelumbinis Semen, Fossilia ossis Mastodi, Lycii radicis Cortex, Achyranthis Radix, Rehmanniae Radix preparata, Perillae Semen, Thujae Semen, Hordei Fructus germinatus, Cuscutae Semen, Morindae Radix, Pini koraiensis Radix and a mixture thereof.

9. A use of a root extract of balloon-flower for enhancing memory in a mammal.

10. The use of claim 9, wherein the mammal is human.

11. The use of claim 9, wherein the balloon-flower is more than 20-year-old long-life balloon-flower.

12. The use of claim 9, wherein the extract is a water extract or an organic solvent extact.

13. The use of claim 12, wherein the water extract is prepared by adding a 5 to 15-fold volume of water to a balloon-flower powder; extracting at 80 to 100° C. for 1 to 24 hours; and filtering the extract thus obtained.

14. The use of claim 9, wherein the extract is administered to the mammal in the form of a composition containing same, said composition being selected from the group consisting of: a pharmaceutical composition, a food composition and a beverage composition.

15. The use of claim 14, wherein the composition further comprises a herb or an extract thereof.

16. The use of claim 15, the herb is selected from the group consisting of Angelicae tenuissimae Radix, Gastrodiae Rhizoma, Bupleuri Radix, Angelicae gigantis Radix, Persicae Semen, Cinnamomi Ramulus, Rhei Rhizoma, Glycyrrhizae Radix, Cnidii Rhizoma, Aurantii nobilis Pericarpium, Alismatis Rhizoma, Coptidis Rhizoma, Scutellariae Radix, Hoelen, Paeoniae Radix, Atractylodis Rhizoma alba, Phellodendri Cortex, Gardeniae Fructus, Pinelliae Tuber, Uncaria Ramulus et Uncus, Ponciri Fructus, Ginseng, Liriopis Tuber, Polygalae Radix, Acori graminei Rhizoma, Atractylodis Rhizoma alba, Chrysanthemi Flos, Ledebouriellae Radix, Zingiberis Rhizoma crudus, Zizyphi Fructus, Salviae Radix, Persicae Semen, Moutan Radicis Cortex, Rehmanniae Radix, Menthae Herba, Dioscoreae Rhizoma, Polyporus, Polygoni multiflori Radix, Allii tuberosi Semen, Cassiae Semen, Lycii Fructus, Araliae cordatae Radix, Eucommiae Cortex, Hedyotis Herba, Saururus Herba, Artemisiae capillaries Herba, Anemarrhenae Rhizoma, Carthami Flos, Astragali Radix, Lycopodium, Ginkgonis Folium, Polygonati Rhizoma, Nelumbinis Semen, Fossilia ossis Mastodi, Lycii radicis Cortex, Achyranthis Radix, Rehmanniae Radix preparata, Perillae Semen, Thujae Semen, Hordei Fructus germinatus, Cuscutae Semen, Morindae Radix, Pini koraiensis Radix and a mixture thereof.
